(19) United States
(12) Patent Application Publication
(10) Pub. No.: US 2008/0050738 A1
(43) Pub. Date: Feb. 28, 2008

(54) DETECTION OF TARGET NUCLEIC ACID

(75) Inventors: Douglas Spencer MILLAR, Revesby (AU); John Robert Melki, Dolls Point (AU); Geoffrey W. Grigg, Linley Point (AU)

Correspondence Address: KNOBBE MARTENS OLSON & BEAR LLP, 2040 MAIN STREET, FOURTEENTH FLOOR, IRVINE, CA, 92614, US

(73) Assignee: HUMAN GENETIC SIGNATURES PTY LTD., Macquarie Park (AU)

(21) Appl. No.: 11/756,534

(22) Filed: May 31, 2007

(30) Foreign Application Priority Data
May 31, 2006 (AU) 2006902955

(51) Int. Cl. C12Q 1/68; C07H 21/04

(52) U.S. Cl. 435/6; 536/23.1; 536/25.3

(57) ABSTRACT

Methods for producing a target nucleic acid molecule from DNA encoding a gene comprising treating DNA from a higher organism with an agent that modifies cytosine to form derivative nucleic acid; and forming a modified nucleic acid having a reduced total number of cytosines compared with the corresponding untreated DNA, wherein the modified nucleic acid molecule includes the target nucleic acid sequence.

### CROSS-REFERENCE TO RELATED APPLICATIONS

The present application claims priority under 35 U.S.C. § 119 to Australian Patent Application No. 2006902955, filed May 31, 2006, the entire contents of which are incorporated herein by reference.

### SEQUENCE LISTING

The present application is being filed along with a Sequence Listing in electronic format. The Sequence Listing is provided as a file entitled ALAR34-001AUS_SeqListing.txt, created on May 31, 2007 which is 3.56 Kb in size. The information in the electronic format of the sequence listing is incorporated herein by reference in its entirety.

### BACKGROUND OF THE INVENTION

1. Field of the Invention

The present invention relates to methods for detecting target nucleic acids.

2. Description of the Related Art

A number of procedures are presently available for the detection of specific nucleic acid molecules. These procedures typically depend on sequence-dependent hybridization between the target DNA (usually genomic or cDNA) and nucleic acid probes which may range in length from short oligonucleotides (20 bases or less) to sequences of many kilobases (kb).

The most widely used method for amplification of specific sequences from within a population of nucleic acid sequences is that of polymerase chain reaction (PCR). In this amplification method, oligonucleotides, generally 20 to 30 nucleotides in length that bind complementary DNA strands and at either end of the region to be amplified, are used to prime DNA synthesis on denatured single-stranded DNA. Successive cycles of denaturation, primer hybridization and DNA strand synthesis using thermostable DNA polymerases allows exponential amplification of the sequences between the primers. RNA sequences can be amplified by first copying using reverse transcriptase to produce a complementary DNA (cDNA) copy. Amplified DNA fragments can be detected by a variety of means including gel electrophoresis, hybridization with labelled probes, use of tagged primers that allow subsequent identification (e.g. by an enzyme linked assay), and use of fluorescently-tagged primers that give rise to a signal upon hybridization with the target DNA (e.g. Beacon and TaqMan systems).

As well as PCR, a variety of other techniques have been developed for detection and amplification of specific nucleotide sequences. One example is the ligase chain reaction. Another example is isothermal amplification which was first described in 1992 and termed Strand Displacement Amplification (SDA). Since then, a number of other isothermal amplification technologies have been described including Transcription Mediated Amplification (TMA) and Nucleic Acid Sequence Based Amplification (NASBA) that use an RNA polymerase to copy RNA sequences but not corresponding genomic DNA.

Other DNA-based isothermal techniques include Rolling Circle Amplification (RCA) in which a DNA polymerase extends a primer directed to a circular template, Ramification Amplification (RAM) that uses a circular probe for target detection and more recently, Helicase-Dependent isothermal DNA amplification (HDA), that uses a helicase enzyme to unwind the DNA strands instead of heat.

Isothermal methods of DNA amplification have also been described. Traditional amplification techniques rely on continuing cycles of denaturation and renaturation of the target molecules at each cycle of the amplification reaction. Heat treatment of DNA results in a certain degree of shearing of DNA molecules, thus when DNA is limiting such as in the isolation of DNA from a small number of cells from a developing blastocyst, or particularly in cases when the DNA is already in a fragmented form, such as in tissue sections, paraffin blocks and ancient DNA samples, this heating-cooling cycle could further damage the DNA and result in loss of amplification signals. Isothermal methods do not rely on the continuing denaturation of the template DNA to produce single stranded molecules to serve as templates from further amplification, but on enzymatic nicking of DNA molecules by specific restriction endonucleases at a constant temperature.

The technique termed Strand Displacement Amplification (SDA) relies on the ability of certain restriction enzymes to nick the unmodified strand of hemi-modified DNA and the ability of a 5′-3′ exonuclease-deficient polymerase to extend and displace the downstream strand. Exponential amplification is then achieved by coupling sense and antisense reactions in which strand displacement from the sense reaction serves as a template for the antisense reaction. Such techniques have been used for the successful amplification of Mycobacterium tuberculosis, HIV-1, Hepatitis C, HPV-16, and Chlamydia trachomatis.

The use of SDA to date has depended on modified phosphorthioate nucleotides in order to produce a hemi-phosphorthioate DNA duplex that on the modified strand would be resistant to enzyme cleavage, resulting in enzymatic nicking instead of digestion to drive the displacement reaction. Recently, however, several “nickase” enzyme have been engineered. These enzymes do not cut DNA in the traditional manner but produce a nick on one of the DNA strands. “Nickase” enzymes include N.Alw1, N.BstNB1 and Mly1. The use of such enzymes would thus simplify the SDA procedure.

In addition, SDA has been improved by the use of a combination of a heat stable restriction enzyme (Ava1) and Heat stable Exo-polymerase (Bst polymerase). This combination has been shown to increase amplification efficiency of the reaction from a 10⁸ fold amplification to 10¹⁰ fold amplification so that it is possible, using this technique, to the amplification of unique single copy molecules. The resultant amplification factor using the heat stable polymerase/enzyme combination is in the order of 10⁹.

To date, all isothermal DNA amplification techniques require the initial double stranded template DNA molecule to be denatured prior to the initiation of amplification. In addition, amplification is only initiated once from each priming event.

For direct detection, the target nucleic acid is most commonly separated on the basis of size by gel electrophoresis and transferred to a solid support prior to hybridization with a probe complementary to the target sequence (Southern and Northern blotting). The probe may be a natural nucleic acid or analogue such as peptide nucleic acid (PNA) or locked nucleic acid (LNA) or intercalating nucleic acid (INA). The probe may be directly labelled (e.g. with ³²P) or an indirect detection procedure may be used. Indirect procedures usually rely on incorporation into the probe of a “tag” such as biotin or digoxigenin and the probe is then detected by means such as enzyme-linked substrate conversion or chemiluminescence.

Another method for direct detection of nucleic acid that has been used widely is “sandwich” hybridization. In this method, a capture probe is coupled to a solid support and the target nucleic acid, in solution, is hybridized with the bound probe. Unbound target nucleic acid is washed away and the bound nucleic acid is detected using a second probe that hybridizes to the target sequences. Detection may use direct or indirect methods as outlined above. Examples of such methods include the “branched DNA” signal detection system, an example that uses the sandwich hybridization principle. A rapidly growing area that uses nucleic acid hybridization for direct detection of nucleic acid sequences is that of DNA micro-arrays. In this process, individual nucleic acid species, that may range from short oligonucleotides, (typically 25-mers in the Affymetrix system), to longer oligonucleotides, (typically 60-mers in the Applied Biosystems and Agilent platforms), to even longer sequences such as cDNA clones, are fixed to a solid support in a grid pattern or photolithographically synthesized on a solid support. A tagged or labelled nucleic acid population is then hybridized with the array and the level of hybridization to each spot in the array quantified. Most commonly, radioactively- or fluorescently-labelled nucleic acids (e.g. cRNAs or cDNAs) are used for hybridization, though other detection systems can be employed, such as chemiluminescence.

A rapidly growing area that uses nucleic acid hybridization for direct detection of nucleic acid sequences is that of DNA micro-arrays. In this process, individual nucleic acid species, that may range from oligonucleotides to longer sequences such as complementary DNA (cDNA) clones, are fixed to a solid support in a grid pattern. A tagged or labelled nucleic acid population is then hybridized with the array and the level of hybridization with each spot in the array quantified. Most commonly, radioactively- or fluorescently-labelled nucleic acids (e.g. cDNAs) were used for hybridization, though other detection systems were employed.

In order to detect target DNA in a sample, it is necessary to design suitable probes or primers that are complementary to regions of interest in a DNA sample. It can be quite difficult or time consuming to prepare the appropriate number probes or primers having necessary nucleotide sequence that will allow the detection of the DNA target but not cross-react with other regions of DNA. It is undesirable to obtain false positives or false negatives in a test.

The present inventors have developed improved methods of forming and detecting target sequences in DNA.

### SUMMARY OF THE INVENTION

The present invention provides a method for detecting a target sequence in DNA encoding a gene, comprising treating DNA from a higher organism with an agent that modifies cytosine to form a derivative nucleic acid; forming a modified nucleic acid from the derivative nucleic acid in which the modified nucleic acid has a different nucleotide sequence from the untreated DNA; and determining the presence of the target sequence by detecting a sequence in the derivative or modified DNA. In one embodiment, the higher organism is an animal. In another embodiment, the higher organism is a human. In one aspect, the treated DNA encodes a gene or forms part of a coding region of DNA. In one embodiment, the target corresponds or relates to one of the following regions of interest in untreated DNA: mutation, alteration, single nucleotide polymorphism, insertion, deletion, rearrangement, tissue typing, species detection, insect typing and other genetic-based targets. In another embodiment, the agent modifies cytosine to uracil to form the derivative nucleic acid. The agent may be bisulfite, acetate or citrate. In one embodiment, the agent is sodium bisulfite. In another embodiment, uracil is replaced as thymine in the modified nucleic acid when the derivative nucleic acid is amplified. The derivative nucleic acid may substantially contain bases adenine (A), guanine (G), thymine (T) and uracil (U) and has substantially the same total number of bases as the corresponding untreated DNA. In one embodiment, the modified nucleic acid is comprised substantially of bases adenine (A), guanine (G) and thymine (T). In another embodiment, the modified nucleic acid is formed by amplifying the derivative nucleic acid. The amplification may be carried out by polymerase chain reaction (PCR), isothermal amplification or signal amplification. In another embodiment, the target nucleic acid molecule is detected by providing a detector ligand capable of binding to the target in the modified nucleic acid molecule and allowing sufficient time for the detector ligand to bind to the target; and measuring binding of the detector ligand to the target to detect the presence of the target.

The present invention also provides a method for producing a target nucleic acid molecule from DNA encoding a gene, comprising treating DNA from a higher organism with an agent that modifies cytosine to form derivative nucleic acid; and forming a modified nucleic acid having a reduced total number of cytosines compared with the corresponding untreated DNA, in which the modified nucleic acid molecule includes the target nucleic acid sequence.

Another embodiment is a method for obtaining a target-specific nucleic acid sequence, comprising obtaining a DNA sequence from a higher organism; forming a modified nucleic acid sequence by carrying out a conversion of the DNA sequence by changing cytosine to uracil or thymine such that the modified nucleic acid sequence comprises substantially no cytosines; and selecting a target-specific nucleic acid sequence from the modified nucleic acid sequence.

The present invention also provides a target having a target-specific nucleic acid sequence obtained by the method described above.

Another embodiment is a method for detecting the presence of a target sequence comprising obtaining DNA from a higher organism; treating the DNA with an agent that modifies cytosine to uracil to form derivative nucleic acid; providing one or more probes capable of binding to a desired target-specific nucleic acid molecule in the derivative nucleic acid; and assaying for the presence of a probe bound to the derivative nucleic acid, wherein detection of bound probe is indicative of the presence of the target

### BRIEF DESCRIPTION OF THE DRAWINGS

FIG. 1 shows the results before and after bisulfite treatment using the 18S rRNA gene to demonstrate a use of the present invention.

FIG. 2 shows the results before and after bisulfite treatment in order to design a single probe that would detect two different SNPs.

### DETAILED DESCRIPTION OF THE INVENTION

The present invention allows a whole new approach to detect DNA that does not require directly targeting the DNA of interest that is present in a sample or occurs naturally in higher organisms. The invention relies on modification of native DNA to form a derivative or modified nucleic acid that does not occur in nature and then detecting the presence of one or more targets, regions or areas of interest in this derivative or modified nucleic acid. As the derivative or modified nucleic acid is formed chemically from the original DNA, information can be obtained indirectly on the naturally occurring DNA without having to probe, bind or directly amplify the native DNA. Additionally, the modification process allows for new nucleic acid sequences, not previously present in nature, to be generated that can be used as targets, probes, etc.

In a general aspect, the present invention relates to modifying nucleotide composition of DNA from higher organisms by treating DNA with an agent that modifies cytosine to uracil, forming modified nucleic acid having a different nucleotide sequence from the untreated DNA but substantially the same overall length and base number, and detecting a sequence in the modified nucleic acid. The present inventors have found that by modifying DNA it is possible to generate new target nucleic acid sequences that are not present in the unmodified DNA but which correspond or relate indirectly to the original DNA.

In a first aspect, the present invention provides a method for producing a target nucleic acid molecule from DNA encoding a gene comprising:

treating DNA from a higher organism with an agent that modifies cytosine to form derivative nucleic acid; and

forming a modified nucleic acid having a reduced total number of cytosines compared with the corresponding untreated DNA, wherein the modified nucleic acid molecule includes the target nucleic acid sequence.

In a second aspect, the present invention provides a method for detecting a target sequence in DNA encoding a gene comprising:

treating DNA from a higher organism with an agent that modifies cytosine to form a derivative nucleic acid;

forming a modified nucleic acid from the derivative nucleic acid, wherein the modified nucleic acid has a different nucleotide sequence from the untreated DNA; and

determining the presence of the target sequence by detecting a sequence in the derivative or modified DNA.

In a third aspect, the present invention provides a method for obtaining a target-specific nucleic acid sequence comprising:

obtaining a DNA sequence from a higher organism;

forming a modified nucleic acid sequence by carrying out a conversion of the DNA sequence by changing cytosine to uracil or thymine such that the modified nucleic acid sequence comprises substantially no cytosines; and

selecting a target-specific nucleic acid sequence from the modified nucleic acid sequence.

In a fourth aspect, the present invention provides a target having a target-specific nucleic acid sequence obtained by the method according to the third aspect of the present invention.

In a fifth aspect, the present invention provides use of the method according to the third aspect of the present invention to obtain probes or primers to bind or amplify the target-specific nucleic acid in a test or assay.

In a sixth aspect, the present invention provides probes or primers obtained by the fifth aspect of the present invention.

In a seventh aspect, the present invention provides a method for detecting the presence of a target sequence comprising:

obtaining DNA from a higher organism;

treating the DNA with an agent that modifies cytosine to form derivative nucleic acid;

providing one or more primers capable of allowing amplification of a desired target-specific nucleic acid molecule to the derivative nucleic acid;

carrying out an amplification reaction on the derivative nucleic acid to form a modified nucleic acid; and

assaying for the presence of an amplified nucleic acid product containing the target sequence, wherein detection of the target sequence is indicative of the presence of the target.

In an eighth aspect, the present invention provides a method for detecting the presence of a target sequence comprising:

obtaining DNA from a higher organism;

treating the DNA with an agent that modifies cytosine to uracil to form derivative nucleic acid;

providing a probe capable of binding to a desired target-specific nucleic acid molecule in the derivative nucleic acid;

assaying for the presence of a probe bound to the derivative nucleic acid, wherein detection of the bound probe is indicative of the presence of the target.

In a ninth aspect, the present invention provides a kit for detecting a target-specific nucleic acid molecule comprising primers or probes according to sixth aspect of the present invention together with one or more reagents or components for an amplification reaction.

In a tenth aspect, the present invention provides a method for amplifying or detecting a target nucleotide sequence in DNA encoding a gene from a higher organism comprising:

obtaining a modified target nucleic acid in which substantially all of the positions naturally occupied by cytosines in the target nucleotide sequence are occupied by a base other than cytosine; and

performing an amplification or hybridization-based detection procedure on the modified target nucleic acid by contacting the modified target nucleic acid with a degenerate probe or primer, the degenerate probe or primer having substantially reduced degree of degeneracy relative to the degree of degeneracy which would be required to amplify or detect the target nucleotide sequence.

In an eleventh aspect, the present invention provides a method for amplifying or detecting a target nucleotide sequence in a sample obtained from a higher organism wherein the target nucleotide sequence naturally occurs in several variant forms, the method comprising:

obtaining a modified target nucleic acid from the sample in which substantially all of the positions naturally occupied by cytosines in the target nucleotide sequence are occupied by a base other than cytosine; and

performing an amplification or hybridization-based detection procedure on the modified target nucleic acid by contacting the modified target nucleic acid with a degenerate probe or primer, the degenerate probe or primer having substantially reduced degree of degeneracy relative to the degree of degeneracy which would be required to amplify or detect the naturally occurring variant forms of the target nucleotide sequence.

Preferably, the higher organism is an animal, more preferably a human. In general, a higher organism is any life form other than a microorganism.

Preferably, the treated DNA encodes a gene or forms part of a coding region of DNA. The target sequence may be any target. Preferably, the target corresponds or relates to a region of interest in untreated DNA such as mutation, alternation, SNP, insertion, deletion, rearrangement, tissue typing, species detection, insect typing or any other genetic-based target.

Preferably, the agent modifies cytosine to uracil to form the derivative nucleic acid.

Preferably, the agent modifies cytosine to uracil which is then replaced as a thymine in the modified nucleic acid when the derivate nucleic acid is amplified. Preferably, the agent used for modifying cytosine is sodium bisulfite. Other agents that similarly modify cytosine, but not methylated cytosine can also be used in the method of the invention. Examples include, but not limited to bisulfite, acetate or citrate. Preferably, the agent is sodium bisulfite, a reagent, which in the presence of water, modifies cytosine into uracil.

Sodium bisulfite (NaHSO₃) reacts readily with the 5,6-double bond of cytosine to form a sulfonated cytosine reaction intermediate which is susceptible to deamination, and in the presence of water gives rise to a uracil sulfite. If necessary, the sulfite group can be removed under mild alkaline conditions, resulting in the formation of uracil. Thus, potentially all cytosines will be converted to uracils. Any methylated cytosines, however, cannot be converted by the modifying reagent due to protection by methylation.

Preferably, the agent modifies a cytosine to a uracil in each strand of complementary double stranded DNA forming two derivative but non-complementary nucleic acid molecules.

Preferably, the derivative nucleic acid has a reduced total number of cytosines compared with the corresponding untreated DNA.

Preferably, the modified nucleic acid has a reduced total number of cytosines compared with the corresponding untreated DNA.

In one preferred form, the derivative nucleic acid substantially contains bases adenine (A), guanine (G), thymine (T) and uracil (U) and has substantially the same total number of bases as the corresponding untreated DNA.

In another preferred form, the modified nucleic acid is comprised substantially of bases adenine (A), guanine (G) and thymine (T).

It will be appreciated that if there is a concern about the presence of methylated cytosines in the DNA, then this methylation can be removed by pre-treating the DNA with a number of rounds of amplification, chemical treatment or enzymatic treatment.

Preferably, the modified nucleic acid is formed by amplifying the derivative nucleic acid. During the amplification process, uracils in the derivative nucleic acid strand are replaced with thymines in the complementary amplified modified nucleic acid strand. Amplification is carried out by any suitable means such as polymerase chain reaction (PCR), isothermal amplification, or signal amplification. In one preferred form, amplification is carried out by PCR. In another preferred form, amplification is carried out by isothermal amplification.

For example, if a target sequence is detected, then it can be inferred that DNA from a higher organism was present in the material being tested.

The present invention can be adapted to replace any present test or genetic assay for targets or regions of DNA. Importantly, the method allows for the alteration of any DNA to a modified nucleic acid and allows new probes or primers to be used as an indirect means of analyzing DNA. Such probes or primers will be different from current probes or primers used for known DNA regions in higher organisms.

The method according to the first aspect of the present invention may further comprise. detecting the target-specific nucleic acid molecule.

In a preferred form, the target nucleic acid molecule is detected by:

providing a detector ligand capable of binding to the target in the modified nucleic acid molecule and allowing sufficient time for the detector ligand to bind to the target; and

measuring binding of the detector ligand to the target to detect the presence of the target.

In another preferred form, the target nucleic acid molecule is detected by separating an amplification product and visualizing the separated product. Preferably, the amplification product is separated by electrophoresis and detected by visualizing one or more bands on a gel.

Preferably, the target nucleic acid molecule does not occur naturally in the higher organism.

In a preferred form of the method according to third aspect of the present invention, modified forms of two or more DNA sequences are obtained and the two or more sequences are compared to obtain at least one nucleic acid containing the target sequence.

In a preferred form of the seventh aspect of the present invention, the nucleic acid molecules are detected by:

providing a detector ligand capable of binding to a region of the nucleic acid molecule and allowing sufficient time for the detector ligand to bind to the region; and

measuring binding of the detector ligand to the nucleic acid molecule to detect the presence of the target.

In another preferred form, the nucleic acid molecules are detected by separating an amplification product and visualizing the separated product.

It will be appreciated that the method according to the third aspect of the present invention can be carried out in silico from known nucleic acid sequences of higher organisms where one or more cytosines in the original sequences is converted to thymine to obtain the modified nucleic acid. Sequence identity can be determined from the converted sequences. Such an in silico method mimics the treatment and amplification steps.

When a target-specific nucleic acid molecule has been obtained for any given organism or gene etc by this method, probes or primers can be designed to ensure amplification of the region of interest in an amplification reaction. Thus, when the probes or primers have been designed, it will be possible to carry out clinical or scientific assays on samples to indirectly detect or analyze DNA of interest.

The target-specific nucleic acid molecule can be unique or have a high degree of similarity within a gene family. One advantage of the present invention is the ability to greatly simplify the potential base differences between, or within, DNA or genes to either an unique molecule or molecules that have close sequence similarity. Specific primers or reduced number of degenerate primers can be used to amplify the target-specific nucleic acid molecule in a given sample.

For double stranded DNA which contains cytosines, the treating step using bisulphate typically results in two derivative nucleic acids (one for each complementary strand), each containing the bases adenine, guanine, thymine and uracil. The two derivative nucleic acids are produced from the two single strands of the double stranded DNA. The two derivative nucleic acids preferably have no cytosines but still have the same total number of bases and sequence length as the original untreated DNA molecule. Importantly, the two derivative nucleic acids are not complimentary to each other and form a top and a bottom strand template for amplification. One or more of the strands can be used to produce the modified nucleic acid molecule. For example, during amplification of the derivative nucleic acids, uracils in the top (or bottom strand) are replaced by thymines in the corresponding amplified modified form of the nucleic acid. As amplification continues, the top (and/or bottom strand if amplified) will be diluted out as each new complimentary strand will have only bases adenine, guanine, thymine.

It will be appreciated that this aspect of the invention also includes nucleic acid molecules having complementary sequences to the target-specific nucleic acid molecule, and nucleic acid molecules that can hybridize, preferably under stringent conditions, to the target-specific nucleic acid molecule.

When a target-specific nucleic acid molecule has been obtained or identified for any given region of DNA, probes or primers can be designed to ensure amplification of the region of interest in an amplification reaction. It is important to note that both strands of a treated and thus converted DNA, (hereafter termed “derivative nucleic acid”) can be analyzed for primer design, since treatment or conversion leads to asymmetries of sequence, and hence different primer sequences are required for the detection of the ‘top’ and ‘bottom’ strands of the same locus, (also known as the ‘Watson’ and ‘Crick’ strands). Thus, there are two populations of molecules, the converted DNA as it exists immediately after conversion, and the population of modified nucleic acid molecules that results from the derivative nucleic acid. Preferably, the derivative nucleic acid is replicated by conventional enzymological means (PCR) or by methods such as isothermal amplification. Primers are typically designed for the converted top strand for convenience but primers can also be generated for the bottom strand. Thus, it will be possible to carry out clinical or scientific assays on samples to detect a given DNA region of interest.

The primers or probes can be designed to allow specific regions of derivative nucleic acid to be amplified. In a preferred form, the primers cause the amplification of the target-specific nucleic acid molecule.

The present invention is suitable for clinical, veterinary, environmental, forensic, scientific, research assays or tests.

The present invention provides a number of advantages over present methods for detection of native or genomic untreated DNA or RNA.

Firstly, forming the derivative DNA reduces the likelihood of the occurrence of secondary structures (like hairpin loops) in single stranded DNA. By reducing risk of secondary structure formation, amplification is more accurate and less likely to generate errors in any amplified products. This is important to reduce risks of misdiagnosis or failure to detect the presence of disease or mutation in a test, for example.

Second, the generation of new target sequences provides the possibility to detect more than one mutation or region of interest with the one probe or primer set in a test.

Third, as the overall degree of complexity of a gene sequence can be simplified, it will be possible to design or require less probes or primers to detect or amplify the sequence of interest.

Fourth, to detect a different targets in the one test, a lower number of probes or primers will be needed.

Fifth, as modified nucleic acid will have an entirely new and unique sequence that does not exist in nature, it should be possible to use that new and sequence information to design improved or competing tests for diseases presently assayed using current technology.

Sixth, the generation of derivative or modified nucleic acid removed ‘GC rich’ regions from DNA to allow more efficient and accurate amplification or probing. AS polymerases can have difficulty in amplifying GC rich regions of DNA, the removal of these regions by the present invention may allow better and more accurate amplification or tests for certain gene mutations.

Seventh, universal primers can be designed for particular genes or conserved genome regions that are common or occur across species using the present invention.

Eighth, the present invention is particularly suited in applications directed to coding regions of a genome. While prior art has concentrated on methylation status of non-coding regions of genomes, the present invention has no relation, application or interest in methylation. Preferably, if there is a concern that the region of interest may have methylation present, it is preferred to remove any methylation of bases before carrying out the present invention. This can be achieved by amplification, chemical treatment or enzymatic treatment of the DNA.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia prior to development of the present invention.

In order that the present invention may be more clearly understood, preferred embodiments will be described with reference to the following drawings and examples.

Definitions

The term “target sequence” as used herein includes any nucleic acid sequence of interest in DNA or modified nucleic acid.

The term “modification” as used herein means DNA (naturally occurring or synthetic) is modified from being comprised of four bases adenine (A), guanine (G), thymine (T) and cytosine (C) to substantially having no cytosine present. In one form, modification results in nucleic acid containing the bases adenine (A), guanine (G), thymine (T) and Uracil (U) but still having substantially the same total number of bases. If the modified sequence undergoes PCR amplification, for example, the modified nucleic acid substantially contains the bases adenine (A), guanine (G), and thymine (T) as each uracil is replaced by thymine in the copied strand.

The term “gene modification” as used herein means the a coding region of DNA (or converted RNA) is modified from being comprised of four bases adenine (A), guanine (G), thymine (T) and cytosine (C) to substantially containing no cytosine but still having substantially the same total number of bases.

The term “genomic modification” as used herein means the genome is modified from being comprised of four bases adenine (A), guanine (G), thymine (T) and cytosine (C) to substantially containing no cytosine but still having substantially the same total number of bases.

The term “derivative nucleic acid ” as used herein means a nucleic acid that substantially contains the bases A, G, T and U (or some other non-A, G or T base or base-like entity) and has substantially the same total number of bases as the corresponding untreated DNA. Preferably, substantially all cytosines in the DNA will have been converted to uracil during treatment with the agent. It will be appreciated that altered cytosines, such as by methylation, may not necessarily be converted to uracil (or some other non-A, G or T base or base-like entity). Preferably, cytosine is modified to uracil.

The term “modified nucleic acid” as used herein means the resulting nucleic acid product obtained from derivative nucleic acid. For example, uracil in the derivative nucleic acid is replaced as a thymine (T) during amplification for example to form the modified nucleic acid molecule. The resulting product has substantially the same number of total bases as the corresponding untreated DNA acid but is substantially made up of a combination of three bases (A, G and T).

The term “modified sequence” as used herein means the resulting nucleic acid sequence of modified nucleic acid obtained from derivative nucleic acid. The resulting modified sequence has substantially the same number of total bases as the corresponding untreated DNA sequence but is substantially made up of a combination of three bases (A, G and T) or (A, G, T and U).

The term “non-modified sequence” as used herein means the nucleic acid sequence of the DNA prior to treatment and modification. A non-converted sequence typically is the sequence of the naturally occurring DNA.

The term “modifies” as used herein means the conversion of a cytosine to another nucleotide. Preferably, the agent modifies cytosine to uracil to form a derivative nucleic acid.

The term “agent that modifies cytosine” as used herein means an agent that is capable of converting cytosine to another chemical entity. Preferably, the agent modifies cytosine to uracil which is then replaced as a thymine to form the modified nucleic acid. Preferably, the agent used for modifying cytosine is sodium bisulfite. Other agents that similarly modify cytosine, but not methylated cytosine can also be used in the method of the invention. Examples include, but not limited to bisulfite, acetate or citrate. Preferably, the agent is sodium bisulfite, a reagent, which in the presence of acidic aqueous conditions, modifies cytosine into uracil. Sodium bisulfite (NaHSO₃) reacts readily with the 5,6-double bond of cytosine to form a sulfonated cytosine reaction intermediate which is susceptible to deamination, and in the presence of water gives rise to a uracil sulfite. If necessary, the sulfite group can be removed under mild alkaline conditions, resulting in the formation of uracil. Thus, potentially all cytosines will be converted to uracils. Any methylated cytosines, however, cannot be converted by the modifying reagent due to protection by methylation. It will be appreciated that cytosine (or any other base) could be modified by enzymatic means to achieve a derivative nucleic acid as taught by the present invention.

There are two broad generic methods by which bases in nucleic acids may be modified: chemical and enzymatic. Thus, modification for the present invention can also be carried out by naturally occurring enzymes, or by yet to be reported artificially constructed or selected enzymes. Chemical treatment, such as bisulfite methodologies, can convert cytosine to uracil via appropriate chemical steps. Similarly, cytosine deaminases, for example, may carry out a conversion to form a derivative nucleic acid. In this early work, cytosine deaminase was not obtained free of other nucleo-deaminases, however, Wang et al. were able to purify such an activity from yeast and E. coli. Thus any enzymatic conversion of cytosine to form a derivative nucleic acid which ultimately results in the insertion of a base during the next replication at that position, that is different to a cytosine, will yield a modified genome. The chemical and enzymatic conversion to yield a derivative followed by a modified genome are applicable to any nucleo-base, be it purines or pyrimidines in naturally occurring DNA.

The term “modified form of the gene or DNA” as used herein means that a gene or DNA, whether naturally occurring or synthetic, which usually contains the four common bases A, G, T and C, consists largely of A, G, T and U as most or all of the Cs in the gene or DNA have been converted to Us by appropriate chemical or enzymatic modification and Ts formation of the modified nucleic acid. During amplification, substantially only three bases A, G and T will be present since Ts replace U's in the corresponding amplified complementary sequence. The modified form after amplification of the derivative gene or DNA means that relative gene or DNA complexity is reduced from a four base foundation towards a three base composition.

The term ‘base-like entity’ as used herein means an entity that is formed by modification of cytosine. A base-like entity can be recognized by a DNA polymerase during amplification of a derivative nucleic acid and the polymerase causes A, G or T to be placed on a newly formed complementary DNA strand at the position opposite the base-like entity in the derivate nucleic acid. Typically, the base-like entity is uracil that has been modified from cytosine in the corresponding untreated DNA. Examples of a base-like entity includes any nucleo-base, be it purine or pyrimidine.

The term “relative complexity reduction” as used herein relates to probe length, namely the increase in average probe length that is required to achieve the same specificity and level of hybridization of a probe to a specific locus, under a given set of molecular conditions in two genes or genomes of the same size, where the first gene or genome is “as is” and consists of the four bases, A, G. T and C, whereas the second gene or genome is of exactly the same length but some cytosines, (ideally all cytosines), have been converted to thymines. The locus under test is in the same location in the original unconverted as well as the converted gene or genome. On average, an 11-mer probe will have a unique location to which it will hybridize perfectly in a genome of 4,194,304 bases consisting of the four bases A, G, T and C, (4¹¹ equals 4,194,304). However, once such a regular genome of 4,194, 304 bases has been converted by bisulfite or other suitable means, this modified genome is now composed of only three bases and is clearly less complex. However the consequence of this decrease in genomic complexity is that our previously unique 11-mer probe no longer has a unique site to which it can hybridize within the modified gene or genome. There are now many other possible equivalent locations of 11 base sequences that have arisen de novo as a consequence of the modification. It will now require a 14-mer probe to find and hybridize to the original locus. Although it may initially appear counter intuitive, one thus requires an increased probe length to detect the original location in what is now a modified three base gene or genome, because more of the gene or genome looks the same, (it has more similar sequences). Thus the reduced relative genomic complexity, (or simplicity of the three base genome), means that one has to design longer probes to find the original unique site.

The term “relative genomic complexity reduction” as used herein can be measured by increased probe lengths capable of being target specific as compared with unmodified DNA. This term also incorporates the type of probe sequences that are used in determining the presence of a DNA target. These probes may have non-conventional backbones, such as those of PNA or LNA or modified additions to a backbone such as those described in INA. Thus, a gene or genome is considered to have reduced relative complexity, irrespective of whether the probe has additional components such as Intercalating pseudonucleotides, such as in INA.

DNA, RNA, locked nucleic acid (LNA), peptide nucleic acid (PNA), MNA, altritol nucleic acid (ANA), hexitol nucleic acid (HNA), intercalating nucleic acid (INA), cyclohexanyl nucleic acid (CNA) and mixtures thereof and hybrids thereof, as well as phosphorous atom modifications thereof, such as but not limited to phosphorothioates, methyl phospholates, phosphoramidites, phosphorodithiates, phosphoroselenoates, phosphotriesters and phosphoboranoates. Non-naturally occurring nucleotides include, but not limited to the nucleotides comprised within DNA, RNA, PNA, INA, HNA, MNA, ANA, LNA, CNA, CeNA, TNA, (2′-NH)-TNA, (3′-NH)-TNA, α-L-Ribo-LNA, α-L-Xylo-LNA, β-D-Xylo-LNA, α-D-Ribo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, β-D-Ribopyranosyl-NA, α-L-Lyxopyranosyl-NA, 2′-R-RNA, α-L-RNA or α-D-RNA, β-D-RNA. In addition non-phosphorous containing compounds may be used for linking to nucleotides such as but not limited to methyliminomethyl, formacetate, thioformacetate and linking groups comprising amides. In particular nucleic acids and nucleic acid analogues may comprise one or more intercalator pseudonucleotides (IPN). The presence of IPN is not part of the complexity description for nucleic acid molecules, nor is the backbone part of that complexity, such as in PNA.

By “INA” is meant an intercalating nucleic acid in accordance with the teaching of WO 03/051901, WO 03/052132, WO 03/052133 and WO 03/052134 (Unest A/S, assigned to Human Genetic Signatures Pty Ltd) incorporated herein by reference. An INA is an oligonucleotide or oligonucleotide analogue comprising one or more intercalator pseudonucleotide (IPN) molecules.

By “HNA” is meant nucleic acids as for example described by Van Aetschot et al., 1995.

By “MNA” is meant nucleic acids as described by Hossain et al, 1998.

“ANA” refers to nucleic acids described by Allert et al, 1999.

“LNA” may be any LNA molecule as described in WO 99/14226 (Exiqon), preferably, LNA is selected from the molecules depicted in the abstract of WO 99/14226. More preferably, LNA is a nucleic acid as described in Singh et al, 1998, Koshkin et al, 1998 or Obika et al., 1997.

“PNA” refers to peptide nucleic acids as for example described by Nielsen et al, 1991.

“Relative complexity reduction” as used herein, does not refer to the order in which bases occur, such as any mathematical complexity difference between a sequence that is ATATATATATATAT (SEQ ID NO: 1) versus one of the same length that is AAAAAAATTTTTTT (SEQ ID NO: 2), nor does it refer to the original re-association data of relative genome sizes (and inferentially, genomic complexities).

“Relative gene or genomic complexity” as used herein refers to an unchanged position of bases in two genes or genomes that is accessed by molecular probes (both the original and modified genes or genomes have bases at invariant positions 1 to n. In the case of the 3 billion base pair haploid human genome of a particular human female, the invariant positions are defined as being from 1 to n, where n is 3,000,000,000. If in the sequence 1 to n, the i^{th} base is a C in the original genome, then the i^{th} base is a T in the converted genome.

The term “genomic DNA” as used herein includes protein encoding nucleic acid, gene encoding nucleic acid, and ribosomal gene regions of eukaryotic organisms.

The term “gene encoding region” as used herein includes any encoding region of DNA or RNA known to the art. A sequence of DNA consisting of a series of nucleotide bases (code) giving rise to the mature messenger RNA that may or may not be translated into the specific amino acids of the protein product.

The term “higher organisms” as used herein includes Kingdom Fungi, Kingdom Plantae and Kingdom Animalia. Typically, the term includes any organism that is not defined as a microorganism herein. The present invention has particular uses in insects and animals including humans.

The term “microorganism” as used herein includes phage, virus, viroid, bacterium, fungus, alga, protozoan, spirochete, single cell organism, or any other microorganism, no matter how variously classified, such as the Kingdom Protoctista by Margulis, L., et al 1990, Handbook of Protoctista, Jones and Bartlett, Publishers, Boston USA, or microorganisms that are associated with humans, as defined in Harrisons Principles of Internal Medicine, 12^{th} Edition, edited by J D Wilson et al., McGraw Hill Inc, as well as later editions. It also includes all microorganisms described in association with human conditions defined in OMIM.

The term “target-specific nucleic acid molecule” as used herein means a nucleic acid which has been determined or obtained using the method according to the present invention which has one or more sequences specific to modified nucleic acid of interest.

The term “close sequence similarity” as used herein includes the above definition of relative sequence complexity and probe lengths as a measure.

Diseases

Examples of diseases that could be detected using the present invention include, but not limited to, Alpha-1-antitrypsin deficiency, Adrenoleukodystrophy Amyotrophic lateral sclerosis, Alzheimer's disease, Ataxia telangiectasia, Becker Muscular Dystrophy, Beta Thalassemia, Central Core Disease, Centronuclear (Myotubular) Myopathy, Cerebellar Ataxia, Chondrodysplasia Punctata, Gaucher disease, Inherited breast and ovarian cancer, Hereditary nonpolyposis colon cancer, Charcot-Marie-Tooth, Congenital adrenal hyperplasia, Cystic fibrosis, Congenital Aganglionic Megacolon, Conradi-Hunnerman Syndrome, Duchenne muscular dystrophy/Becker muscular dystrophy, Dystonia, Fanconi anemia group C, Factor V-Leiden, Factor VIII Deficiency, Factor IX Deficiency, Fragile X syndrome, Familial Spastic Paraparesis, Friedrich's Ataxia, Gardener Syndrome, Glycogen Storage Disease, Happle Syndrome, Hemophilia, Hereditary Motor-Sensory Neuropathy, Hereditary Spastic Paraplegia, Hers Disease, Hirschsprung Disease, Hypoxanthine-Guanine, Phosphoribosyl Transferase (HPRT) Deficiency, Hemophilia A and B, Hereditary Hemochromatosis, Huntington's disease, Ichthyosis, Ichthyosis Follicularis, Atrichia and Photophobia Syndrome, Ichthyosis, Hepatosplenomegaly, Cerebellar Degeneration, Ichthyosis, Follicular Atrophoderma Hypotrichosis, Ichthyosis, Follicular Atrophoderina Hypohidrosis, Kallman Syndrome, Kelley-Seegmiller Syndrome, Kennedy Disease, Lou Gehrig's Disease, Mitochondrial Myopathy, Myopathies, Myotonia Congenita, Myotubular Myopathy Myotonic dystrophy, Neurofibromatosis type 1, Nemaline Myopathy, Nephrolithiasis, Paramyotonia Congenita, Parkinson's Disease, Periodic Paralysis, Peroneal Muscle Atrophy, Polycystic Ovary Syndrome, Prostate Cancer, Phenylketonuria, Adult Polycystic Kidney Disease, Prader Willi/Angelman syndromes, Retinitis Pigmentosa, Spinal and Bulbar Muscular Atrophy, Stein-Leventhal Syndrome, Strumpell Disease, Sickle cell disease, Spinocerebellar ataxia, type 1, Spinal muscular atrophy, Thalassemias, Tay-Sachs Disease, Lesch-Nyhan Syndrome, Thrombocytopenia and Von Willebrand Disease

### Materials and Methods

Extraction of DNA

In general, DNA can be obtained from any suitable source. Examples include, but not limited to, environmental samples, clinical samples, bodily fluids, liquid samples, solid samples such as tissue. DNA from samples can be obtained by standard procedures. An example of a suitable extraction is as follows. The sample of interest is placed in 400 μl of 7 M Guanidinium hydrochloride, 5 mM EDTA, 100 mM Tris/HCl pH 6.4, 1% Triton-X-100, 50 mM Proteinase K (Sigma), 100 μg/ml yeast tRNA. The sample is thoroughly homogenized with disposable 1.5 ml pestle and left for 48 hours at 60° C. After incubation the sample is subjected to five freeze/thaw cycles of dry ice for 5 minutes/95° C. for 5 minutes. The sample is then vortexed and spun in a microfuge for 2 minutes to pellet the cell debris. The supernatant is removed into a clean tube, diluted to reduce the salt concentration then phenol:chloroform extracted, ethanol precipitated and resuspended in 50 μl of 10 mM Tris/0.1 mM EDTA.

General DNA or RNA Extraction

Any suitable method for obtaining nucleic acid material can be used. Examples include, but are not limited to, commercially available DNA/RNA kits or reagents, workstation, standard cell lysis buffers containing protease reagents and organic extraction procedures, which are well known to those of skill in the art.

DNA extraction from Cytology samples from patients.
a) The sample was shaken vigorously by hand to resuspend any sedimented cells and to ensure the homogeneity of the solution.
b) 4 ml of the resuspended cells were transferred to a 15 ml Costar centrifuge tube.
c) The tubes were centrifuged in a swing-out bucket rotor at 3000×g for 15 minutes.
d) The supernatant was carefully decanted and discarded without disturbing the pelleted cellular material.
e) The pelleted cells were resuspended in 200 μl of lysis buffer (100 mM Tris/HCl pH 8.0, 2 mM EDTA pH 8.0, 0.5% SDS, 0.5% Triton-X-100) and mixed well until the solution was homogeneous.
f) 80 μl of the sample was transferred to a 96 well sample preparation plate
g) 20 μl of Proteinase K was added and the solution incubated at 55° C. for 1 hour (this procedure results in cell lysis)
   Bisulfite Treatment of DNA Samples

Bisulfite treatment was carried out according the MethylEasy™ High Throughput DNA bisulfite modification kit (Human Genetic Signatures Pty Ltd, Australia) see also below.

Surprisingly, it has been found by the present inventors that there is no need to separate the DNA of interest from other sources of nucleic acids, for example when there is microbial DNA in a sample of human cells. The treatment step can be used for an vast mixture of different DNA types and yet a target-specific nucleic acid can be still identified by the present invention. It is estimated that the limits of detection in a complex DNA mixtures are that of the limits of standard PCR detection which can be down to a single copy of a target nucleic acid molecule.

Samples

Any suitable sample can be used for the present invention. Examples include, but not limited to, cell culture, clinical samples, veterinary samples, biological fluids, forensic samples, tissue culture samples, environmental samples, water samples, effluent. As the present invention is adaptable for detecting or testing DNA or RNA from any source, this list should not be considered as exhaustive.

Kits

The present invention can be implemented in the form of various kits, or combination of kits and instantiated in terms of manual, semi automated or fully robotic platforms. In a preferred form, the MethylEasy™ kit (Human Genetic Signatures Pty Ltd, Australia) allow modification of DNA in 96 or 384 plates using a robotic platform such as EpMotion.

Bisulfite Treatment

An exemplary protocol for effective bisulfite treatment of nucleic acid is set out below. The protocol results in retaining substantially all DNA treated. This method is also referred to herein as the Human Genetic Signatures (HGS) method. It will be appreciated that the volumes or amounts of sample or reagents can be varied.

Preferred method for bisulfite treatment can be found in U.S. Ser. No. 10/428310 or PCT/AU20041000549 (Human Genetic Signatures Pty Ltd, Australia) incorporated herein by reference.

To 2 μg of DNA, which can be pre-digested with suitable restriction enzymes if so desired, 2 μl ( 1/10 volume) of 3 M NaOH (6 g in 50 ml water, freshly made) was added in a final volume of 20 μl. This step denatures the double stranded DNA molecules into a single stranded form, since the bisulfite reagent preferably reacts with single stranded molecules. The mixture was incubated at 37° C. for 15 minutes. Incubation at temperatures above room temperature can be used to improve the efficiency of denaturation.

After the incubation, 208 μl 2 M Sodium Metabisulfite (7.6 g in 20 ml water with 416 ml 10 N NaOH; BDH AnalaR #10356.4D; freshly made) and 12 μl of 10 mM Quinol (0.055 g in 50 ml water, BDH AnalR #103122E; freshly made) were added in succession. Quinol is a reducing agent and helps to reduce oxidation of the reagents. Other reducing agents can also be used, for example, dithiothreitol (DTT), mercaptoethanol, quinone (hydroquinone), or other suitable reducing agents. The sample was overlaid with 200 μl of mineral oil. The overlaying of mineral oil prevents evaporation and oxidation of the reagents but is not essential. The sample was then incubated overnight at 55° C. Alternatively the samples can be cycled in a thermal cycler as follows: incubate for about 4 hours or overnight as follows: Step 1, 55° C./2 hr cycled in PCR machine; Step 2, 95° C./2 min. Step 1 can be performed at any temperature from about 37° C. to about 90° C. and can vary in length from 5 minutes to 8 hours. Step 2 can be performed at any temperature from about 70° C. to about 99° C. and can vary in length from about 1 second to 60 minutes, or longer.

After the treatment with Sodium Metabisulfite, the oil was removed, and 1 μl tRNA (20 mg/ml) or 2 μl glycogen were added if the DNA concentration was low. These additives are optional and can be used to improve the yield of DNA obtained by co-precipitating with the target DNA especially when the DNA is present at low concentrations. The use of additives as carrier for more efficient precipitation of nucleic acids is generally desired when the amount nucleic acid is <0.5 μg.

An isopropanol cleanup treatment was performed as follows: 800 μl of water were added to the sample, mixed and then 1 ml isopropanol was added. The water or buffer reduces the concentration of the bisulfite salt in the reaction vessel to a level at which the salt will not precipitate along with the target nucleic acid of interest. The dilution is generally about ¼ to 1/1000 so long as the salt concentration is diluted below a desired range, as disclosed herein.

The sample was mixed again and left at 4° C. for a minimum of 5 minutes. The sample was spun in a microfuge for 10-15 minutes and the pellet was washed 2× with 70% ETOH, vortexing each time. This washing treatment removes any residual salts that precipitated with the nucleic acids.

The pellet was allowed to dry and then resuspended in a suitable volume of T/E (10 mM Tris/0.1 mM EDTA) pH 7.0-12.5 such as 50 μl. Buffer at pH 10.5 has been found to be particularly effective. The sample was incubated at 37° C. to 95° C. for 1 min to 96 hr, as needed to suspend the nucleic acids.

Another example of bisulfite treatment can be found in WO 2005021778 (incorporated herein by reference) which provides methods and materials for conversion of cytosine to uracil. In some embodiments, a nucleic acid, such as gDNA, is reacted with bisulfite and a polyamine catalyst, such as a triamine or tetra-amine. Optionally, the bisulfite comprises magnesium bisulfite. In other embodiments, a nucleic acid is reacted with magnesium bisulfite, optionally in the presence of a polyamine catalyst and/or a quaternary amine catalyst. Also provided are kits that can be used to carry out methods of the invention. It will be appreciated that these methods would also be suitable for the present invention in the treating step.

Amplification

PCR amplifications were performed in 25 μl reaction mixtures containing 2 μl of bisulfite-treated DNA, using the Promega PCR master mix, 6 ng/μl of each of the primers. Strand-specific nested primers can be used for amplification. 1^{st} round PCR amplifications were carried out using PCR primers 1 and 4 (see below). Following 1^{st} round amplification, 1 μl of the amplified material was transferred to 2^{nd} round PCR premixes containing PCR primers 2 and 3 and amplified as previously described. Samples of PCR products were amplified in a ThermoHybaid PX2 thermal cycler under the conditions: 1 cycle of 95° C. for 4 minutes, followed by 30 cycles of 95° C. for 1 minute, 50° C. for 2 minutes and 72° C. for 2 minutes; 1 cycle of 72° C. for 10 minutes. 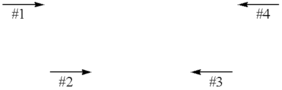
Multiplex Amplification

If multiplex amplification is required for detection, the following methodology can be carried out.

One μl of bisulfite treated DNA is added to the following components in a 25 μl reaction volume, ×1 Qiagen multiplex master mix, 5-100 ng of each 1^{st} round INA or oligonucleotide primer 1.5-4.0 mM MgSO₄, 400 uM of each dNTP and 0.5-2 unit of the polymerase mixture. The components are then cycled in a hot lid thermal cycler as follows. Typically there can be up to 200 individual primer sequences in each amplification reaction.

| | | | |
|---|---|---|---|
| Step 1 | 94° C. | 15 minute | 1 cycle |
| Step 2 | 94° C. | 1 minute | |
| | 50° C. | 3 minutes | 35 cycles |
| | 68° C. | 3 minutes | |
| Step 3 | 68° C. | 10 minutes | 1 cycle |

A second round amplification is then performed on a 1 μl aliquot of the first round amplification that is transferred to a second round reaction tube containing the enzyme reaction mix and appropriate second round primers. Cycling is then performed as above.

Primers

Any suitable PCR or isothermal primers can be used for the present invention. A primer typically has a complementary sequence to a sequence which will be amplified. Primers are typically oligonucleotides but can be oligonucleotide analogues.

Probes

The probe may be any suitable nucleic acid molecule or nucleic acid analogue. Examples include, but not limited to, DNA, RNA, locked nucleic acid (LNA), peptide nucleic acid (PNA), MNA, altritol nucleic acid (ANA), hexitol nucleic acid (HNA), intercalating nucleic acid (INA), cyclohexanyl nucleic acid (CNA) and mixtures thereof and hybrids thereof, as well as phosphorous atom modifications thereof, such as but not limited to phosphorothioates, methyl phospholates, phosphoramidites, phosphorodithiates, phosphoroselenoates, phosphotriesters and phosphoboranoates. Non-naturally occurring nucleotides include, but not limited to the nucleotides comprised within DNA, RNA, PNA, INA, HNA, MNA, ANA, LNA, CNA, CeNA, TNA, (2′-NH)-TNA, (3′-NH)-TNA, α-L-Ribo-LNA, α-L-Xylo-LNA, β-D-Xylo-LNA, α-D-Ribo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, β-D-Ribopyranosyl-NA, α-L-Lyxopyranosyl-NA, 2′-R-RNA, α-L-RNA or α-D-RNA, β-D-RNA. In addition non-phosphorous containing compounds may be used for linking to nucleotides such as but not limited to methyliminomethyl, formacetate, thioformacetate and linking groups comprising amides. In particular nucleic acids and nucleic acid analogues may comprise one or more intercalator pseudonucleotides.

Preferably, the probes are DNA or DNA oligonucleotides containing one or more internal IPNs forming INA.

Electrophoresis

Electrophoresis of samples was performed according to the E-gel system user guide (Invitrogen).

Detection Methods

Numerous possible detection systems exist to determine the status of the desired sample. It will be appreciated that any known system or method for detecting nucleic acid molecules could be used for the present invention. Detection systems include, but not limited to:
I. Hybridization of appropriately labeled DNA to a micro-array type device which could select for 10→200,000 individual components. The arrays could be composed of either INAs, PNAs or nucleotide or modified nucleotides arrays onto any suitable solid surface such as glass, plastic, mica, nylon, bead, magnetic bead, fluorescent bead or membrane;
II. Southern blot type detection systems;
III. Standard PCR detection systems such as agarose gel, fluorescent read outs such as Genescan analysis. Sandwich hybridization assays, DNA staining reagents such as ethidium bromide, Syber green, antibody detection, ELISA plate reader type devices, fluorimeter devices;
IV. Real-Time PCR quantitation of specific or multiple genomic amplified fragments or any variation on that;
V. Any of the detection systems outlined in the WO 2004/065625 such as fluorescent beads, enzyme conjugates, radioactive beads and the like;
VI. Any other detection system utilizing an amplification step such as ligase chain reaction or Isothermal DNA amplification technologies such as Strand Displacement Amplification (SDA);
VII. Multi-photon detection systems;
VIII. Electrophoresis and visualization in gels; and
IX. Any detection platform used or could be used to detect nucleic acid.
   Intercalating Nucleic Acids

Intercalating nucleic acids (INA) are non-naturally occurring polynucleotides which can hybridize to nucleic acids (DNA and RNA) with sequence specificity. INA are candidates as alternatives/substitutes to nucleic acid probes in probe-based hybridization assays because they exhibit several desirable properties. INA are polymers which hybridize to nucleic acids to form hybrids which are more thermodynamically stable than a corresponding naturally occurring nucleic acid/nucleic acid complex. They are not substrates for the enzymes which are known to degrade peptides or nucleic acids. Therefore, INA should be more stable in biological samples, as well as, have a longer shelf-life than naturally occurring nucleic acid fragments. Unlike nucleic acid hybridization which is very dependent on ionic strength, the hybridization of an INA with a nucleic acid is fairly independent of ionic strength and is favored at low ionic strength under conditions which strongly disfavor the hybridization of naturally occurring nucleic acid to nucleic acid. The binding strength of INA is dependent on the number of intercalating groups engineered into the molecule as well as the usual interactions from hydrogen bonding between bases stacked in a specific fashion in a double stranded structure. Sequence discrimination is more efficient for INA recognizing DNA than for DNA recognizing DNA.

Preferably, the INA is the phosphoramidite of (S)-1-O-(4,4′-dimethoxytriphenylmethyl)-3-O-(1-pyrenylmethyl)-glycerol.

INA are synthesized by adaptation of standard oligonucleotide synthesis procedures in a format which is commercially available. Full definition of INA and their synthesis can be found in WO 03/051901, WO 03/052132, WO 03/052133 and WO 03/052134 (Unest A/S, assigned to Human Genetic Signatures Pty Ltd Australia) incorporated herein by reference.

There are indeed many differences between INA probes and standard nucleic acid probes. These differences can be conveniently broken down into biological, structural, and physico-chemical differences. As discussed above and below, these biological, structural, and physico-chemical differences may lead to unpredictable results when attempting to use INA probes in applications were nucleic acids have typically been employed. This non-equivalency of differing compositions is often observed in the chemical arts.

With regard to biological differences, nucleic acids are biological materials that play a central role in the life of living species as agents of genetic transmission and expression. Their in vivo properties are fairly well understood. INA, however, is a recently developed totally artificial molecule, conceived in the minds of chemists and made using synthetic organic chemistry. It has no known biological function.

Structurally, INA also differs dramatically from nucleic acids. Although both can employ common nucleobases (A, C, G, T, and U), the composition of these molecules is structurally diverse. The backbones of RNA, DNA and INA are composed of repeating phosphodiester ribose and 2-deoxyribose units. INA differ from DNA or RNA in having one or more large flat molecules attached via a linker molecule(s) to the polymer. The flat molecules intercalate between bases in the complementary DNA stand opposite the INA in a double stranded structure.

The physico/chemical differences between INA and DNA or RNA are also substantial. INA binds to complementary DNA more rapidly than nucleic acid probes bind to the same target sequence. Unlike DNA or RNA fragments, INA bind poorly to RNA unless the intercalating groups are located in terminal positions. Because of the strong interactions between the intercalating groups and bases on the complementary DNA strand, the stability of the INA/DNA complex is higher than that of an analogous DNA/DNA or RNA/DNA complex.

Unlike other nucleic acids such as DNA or RNA fragments or PNA, INA do not exhibit self aggregation or binding properties.

As INA hybridize to nucleic acids with sequence specificity, INA are useful candidates for developing probe-based assays and are particularly adapted for kits and screening assays. INA probes, however, are not the equivalent of nucleic acid probes. Consequently, any method, kits or compositions which could improve the specificity, sensitivity and reliability of probe-based assays would be useful in the detection, analysis and quantitation of DNA containing samples. INA have the necessary properties for this purpose.

### Results

Disease Detection

The sample can be prepared from tissue, cells or can be any biological sample such as blood, urine, feces, semen, cerebrospinal fluid, lavage, cells or tissue from sources such as brain, colon, urogenital, lung, renal, hematopoietic, breast, thymus, testis, ovary, uterus, tissues from embryonic or extra-embryonic lineages, environmental samples, plants, microorganisms including bacteria, intracellular parasites virus, fungi, protozoan, viroid and the like. The best described mammalian cell types suitable for treatment by the present invention are summarized in B. Alberts et al., 1989, The Molecular Biology of the Cell, 2^{nd} Edition, Garland Publishing Inc New York and London, pp 995-997.

The analyses are meant to include the naturally occurring variation between cells, tissues and organs of healthy individuals, (health as defined by the WHO), as well as cells, tissues and organs from diseased individuals. Diseased in this sense includes all human diseases, afflictions, ailments and deviant conditions described or referred to in Harrison's Principles of Internal Medicine, 12th Edition, edited by Jean D Wilson et al., McGrraw Hill Inc, and subsequent later editions; as well as all diseases, afflictions ailments and deviant conditions described in OMIM, but with emphases on the leading causes of death, namely, malignant neoplasms, (cancer), ischemic heart disease, cerebrovascular disease, chronic obstructive pulmonary disease, pneumonia and influenza, diseases of arteries, (including atherosclerosis and aortic aneurysm), diabetes mellitus, and central nervous system diseases, together with socially debilitating conditions such as anxiety, stress related neuropsychiatric conditions and obesity, and all conditions arising from abnormal chromosome number or chromosome rearrangements, (aneuploidy involving autosomes as well as sex chromosomes, duplications, deficiencies, translocations and insertions), as well as similar abnormalities of the mitochondrial genomes.

The normal or diseased individuals may be from (i) populations of diverse ethnicity and evolutionary lineages; (ii) strains and geographical isolates; (iii) sub species; (iv) twins or higher order multiplets of the same or different sex; (v) individuals arising from normal methods of conjugation, artificial insemination, cloning by embryonic stem cell methods, or by nuclear transfer, (from somatic or germ line nuclei), or from the input or modification of mitochondrial or other cellular organelles; (vi) individuals deriving from transgenic knock-out, knock-in or knock-down methods, (either in vivo, ex vivo, or by any method in which gene activity is transiently or permanently altered, e.g., by RNAi, ribozyme, transposon activation, drug or small molecule methodologies, Peptide Nucleic Acid (PNA), Intercalating Nucleic Acid (INA), Altritol Nucleic Acid (ANA), Hexitol Nucleic Acid (HNA), Locked Nucleic Acid (LNA), Cyclohexanyl Nucleic Acid (CNA), and the like, or nucleic acid based conjugates, including but not restricted to Trojan peptides, or individuals at any stages of pregnancy, normal or ectopic.

The analyses also include DNA or RNA from prokaryotic or eukaryotic organisms and viruses (or combinations thereof), that are associated with human diseases in extracellular or intracellular modes, for the purposes of determining, and therapeutically altering, in both normally varying and diseased systems, the changed parameters and underlying mechanisms of:
(i) genetic diseases;
(ii) non-genetic or epigenetic diseases caused by environmentally induced factors, be they of biological or non-biological origin, (environmental in this sense being taken to also include the environment within the organism itself, during all stages of pregnancy, or under conditions of fertility and infertility treatments);
(iii) predisposition to genetic or non genetic diseases, including effects brought about by the “prion” class of factors, by exposure to pressure changes and weightlessness, or by radiation effects;
   Trinucleotide Repeat Diseases

A recognized problem with PCR amplification of genes involved in trinucleotide repeat diseases is PCR stutter. A specific sequence and/or secondary structure and high CG content appear to be requirements for this slippage mechanism.

By reducing the CG content and or secondary structure of the repeat, bisulfite genomic simplification will reduce the problem of stutter and assist in the more reliable method for determining the true number of repeat units in people suffering from tri-nucleotide repeat diseases

In the case of FRAXE which is caused by the FMR2 gene on the X-chromosome, the normal FMR2 allele has between 6 and 35 copies of GCC, however, in people with the disorder the allele has over 200 copies. After simplification this repeat will now be read as GTT. Thus the polymerase enzyme should be less prone to slippage and thus a more accurate representation of expansion length should be generated.

Examples of human diseases caused by trinucleotide expansion can be seen below:
DRPLA (Dentatorubropallidoluysian atrophy, CAG), HD (Huntington's disease, CAG)
SBMA (Spinobulbar muscular atrophy or Kennedy disease, CAG)
SCA1 (Spinocerebellar ataxia Type 1, SCA2 (Spinocerebellar ataxia Type 2)
SCA3 (Spinocerebellar ataxia Type 3 or Machado-Joseph Disease, SCA6 (Spinocerebellar ataxia Type 6), SCA7 (Spinocerebellar ataxia Type 7)
FRAXA (Fragile X syndrome, CCG)
FRAXE (Fragile XE mental retardation, GCC)
FRDA (Friedreich's ataxia, GAA), DM (Myotonic dystrophy, CTG)
SCA8 (Spinocerebellar ataxia Type 8, CTG), SCA12 (Spinocerebellar ataxia Type 12, CAG)
   Genetic Simplification

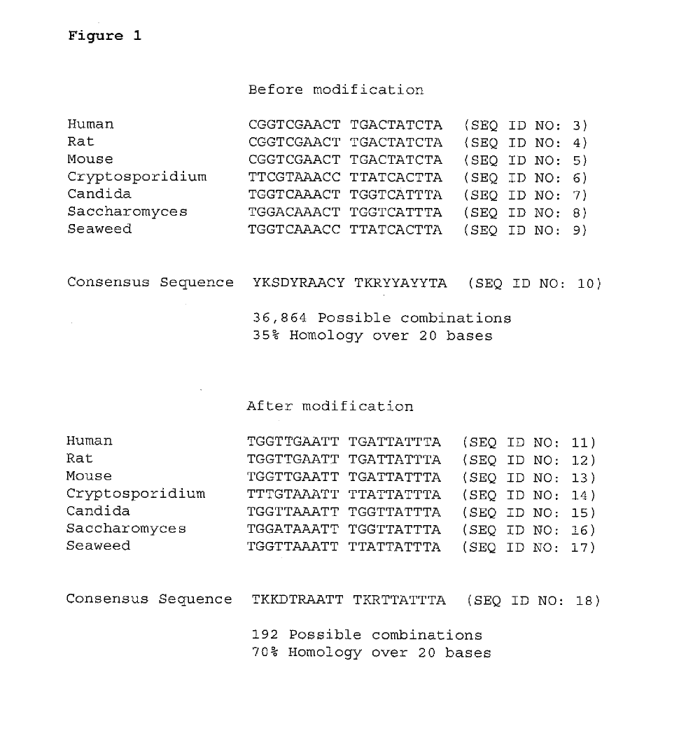

FIG. 1 shows the results before and after bisulfite treatment using the 18S rRNA gene. Seven 18S rRNA genomic sequences from a diverse range of organisms from human to fungi were used to demonstrate the bisulfite genomic simplification technique. Before simplification a total of 36,864 combinations of primer would be required to universally detect all of the above organisms. After treatment, only 192 combinations of primer (a 192 fold simplification) would be required to detect the same seven organisms. SNP detection

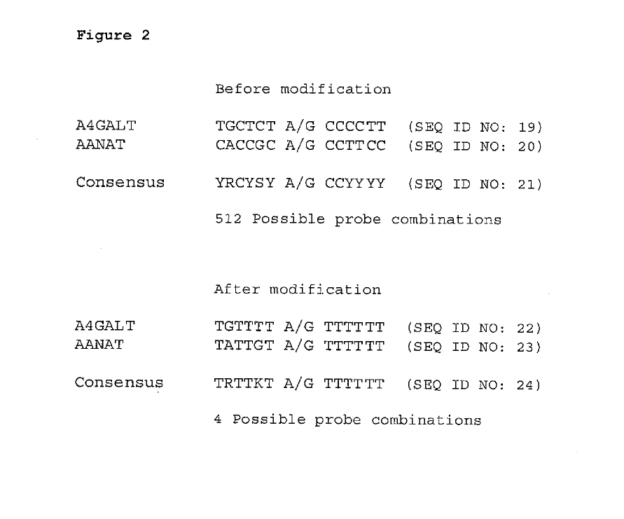

FIG. 2 shows the results before and after bisulfite treatment in order to design a single probe that would detect two different SNPs. As can be seen before treatment, 512 probes would be required to detect both SNPs while after treatment only 4 probes would be required to detect the same SNPs.

Thus using bisulfite treatment it is possible to design probes that detect multiple SNPs on different genes thus greatly reducing the cost and level of multiplexing required for global SNP analysis.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

What is claimed is:

1. A method for detecting a target sequence in DNA encoding a gene, comprising:
treating DNA from a higher organism with an agent that modifies cytosine to form a derivative nucleic acid;
forming a modified nucleic acid from the derivative nucleic acid, wherein the modified nucleic acid has a different nucleotide sequence from the untreated DNA; and
determining the presence of the target sequence by detecting a sequence in the derivative or modified DNA.

2. The method according to claim 1, wherein the higher organism is an animal.

3. The method according claim 2, wherein the higher organism is a human.

4. The method according to claim 1, wherein the treated DNA encodes a gene or forms part of a coding region of DNA.

5. The method according to claim 1, wherein the target corresponds or relates to a region of interest in untreated DNA selected from the group consisting of mutation, alternation, SNP, insertion, deletion, rearrangement, tissue typing, species detection, insect typing and other genetic-based targets.

6. The method according to claim 1, wherein the agent modifies cytosine to uracil to form the derivative nucleic acid.

7. The method according to claim 6, wherein the agent is bisulfite, acetate or citrate.

8. The method according to claim 7, wherein the agent is sodium bisulfite.

9. The method according to claim 6, wherein uracil is replaced as thymine in the modified nucleic acid when the derivate nucleic acid is amplified.

10. The method according to claim 1, wherein the derivative nucleic acid substantially contains bases adenine (A), guanine (G), thymine (T) and uracil (U) and has substantially the same total number of bases as the corresponding untreated DNA.

11. The method according to claim 1, wherein the modified nucleic acid is comprised substantially of bases adenine (A), guanine (G) and thymine (T).

12. The method according to claim 1, wherein the modified nucleic acid is formed by amplifying the derivative nucleic acid.

13. The method according to claim 12, wherein amplification is carried out by polymerase chain reaction (PCR), isothermal amplification, or signal amplification.

14. The method according to claim 15 wherein the target nucleic acid molecule is detected by:
providing a detector ligand capable of binding to the target in the modified nucleic acid molecule and allowing sufficient time for the detector ligand to bind to the target; and
measuring binding of the detector ligand to the target to detect the presence of the target.

15. A method for producing a target nucleic acid molecule from DNA encoding a gene, comprising:
treating DNA from a higher organism with an agent that modifies cytosine to form derivative nucleic acid; and
forming a modified nucleic acid having a reduced total number of cytosines compared with the corresponding untreated DNA, wherein the modified nucleic acid molecule includes the target nucleic acid sequence.

16. A method for obtaining a target-specific nucleic acid sequence, comprising:
obtaining a DNA sequence from a higher organism;
forming a modified nucleic acid sequence by carrying out a conversion of the DNA sequence by changing cytosine to uracil or thymine such that the modified nucleic acid sequence comprises substantially no cytosines; and
selecting a target-specific nucleic acid sequence from the modified nucleic acid sequence.

17. A target having a target-specific nucleic acid sequence obtained by the method according to claim 16.

18. A method for detecting the presence of a target sequence, comprising:
obtaining DNA from a higher organism;
treating the DNA with an agent that modifies cytosine to form derivative nucleic acid;
providing one or more primers capable of allowing amplification of a desired target-specific nucleic acid molecule to the derivative nucleic acid;
carrying out an amplification reaction on the derivative nucleic acid to form a modified nucleic acid; and
assaying for the presence of an amplified nucleic acid product containing the target sequence, wherein detection of the target sequence is indicative of the presence of the target.

19. A method for detecting the presence of a target sequence, comprising:
obtaining DNA from a higher organism;
treating the DNA with an agent that modifies cytosine to uracil to form derivative nucleic acid;
providing one or more probes capable of binding to a desired target-specific nucleic acid molecule in the derivative nucleic acid; and
assaying for the presence of a probe bound to the derivative nucleic acid, wherein detection of bound probe is indicative of the presence of the target.


SEQUENCE LISTING (24 sequences)

SEQ ID NO: 1 - DNA, 14 bp, human
atatatatatatat

SEQ ID NO: 2 - DNA, 14 bp, human
aaaaaaattttttt

SEQ ID NO: 3 - DNA, 20 bp, human
cggtcgaacttgactatcta

SEQ ID NO: 4 - DNA, 20 bp, rat
cggtcgaacttgactatcta

SEQ ID NO: 5 - DNA, 20 bp, mouse
cggtcgaacttgactatcta

SEQ ID NO: 6 - DNA, 20 bp, bacterial
ttcgtaaaccttatcactta

SEQ ID NO: 7 - DNA, 20 bp, fungal
tggtcaaacttggtcattta

SEQ ID NO: 8 - DNA, 20 bp, yeast
tggacaaacttggtcattta

SEQ ID NO: 9 - DNA, 20 bp, plant
tggtcaaaccttatcactta

SEQ ID NO: 10 - DNA, 20 bp, none
yksdyraacytkryyayyta

SEQ ID NO: 11 - DNA, 20 bp, human
tggttgaatttgattattta

SEQ ID NO: 12 - DNA, 20 bp, rat
tggttgaatttgattattta

SEQ ID NO: 13 - DNA, 20 bp, mouse
tggttgaatttgattattta

SEQ ID NO: 14 - DNA, 20 bp, bacterial
tttgtaaattttattattta

SEQ ID NO: 15 - DNA, 20 bp, fungal
tggttaaatttggttattta

SEQ ID NO: 16 - DNA, 20 bp, yeast
tggataaatttggttattta

SEQ ID NO: 17 - DNA, 20 bp, plant
tggttaaattttattattta

SEQ ID NO: 18 - DNA, 20 bp, none
tkkdtraatttkrttattta

SEQ ID NO: 19 - DNA, 14 bp, human
tgctctagcccctt

SEQ ID NO: 20 - DNA, 14 bp, none
caccgcagccttcc

SEQ ID NO: 21 - DNA, 14 bp, none
yrcysyagccyyyy

SEQ ID NO: 22 - DNA, 14 bp, human
tgttttagtttttt

SEQ ID NO: 23 - DNA, 14 bp, human
tattgtagtttttt

SEQ ID NO: 24 - DNA, 14 bp, none
trttktagtttttt